(19) United States
(12) Patent Application Publication
(10) Pub. No.: US 2014/0364898 A1
(43) Pub. Date: Dec. 11, 2014

(54) NOVEL METHODS AND DEVICES FOR THE THERAPEUTIC TREATMENT OF MYOPIA

(71) Applicant: Lawrence Shengzhe Wang, Knoxville, TN (US)

(72) Inventor: Lawrence Shengzhe Wang, Knoxville, TN (US)

(21) Appl. No.: 14/292,233

(22) Filed: May 30, 2014

Related U.S. Application Data
(60) Provisional application No. 61/831,868, filed on Jun. 6, 2013.

(51) Int. Cl. A61F 9/04

(52) U.S. Cl. CPC A61F 9/04
USPC 606/204.25

(57) ABSTRACT

Non-surgical therapeutic methods and devices for the treatment of myopia are presented. The eyeball of a patient suffering from myopia is elongated causing refracted light to fall onto a spot in-front of the retina. Studies have shown that the reshaping of the cornea (i.e. through contact lenses or laser surgery) improves the vision of patients. Methods and devices proposed herein mainly target restoring the proper eyeball shape in order to allow the refracted light to fall on the retina, and providing a treatment regime for a patient suffering from myopia to apply a mild but sustained pressure to one or two eyeballs for a sustained period of time. The patient may repeat this process multiple times as needed until their vision improves.

### CROSS REFERENCE TO RELATED APPLICATION

The present application claims priority to co-pending U.S. provisional patent application No. 61/831,868 filed Jun. 6, 2013, and entitled “NOVEL METHODS AND DEVICES FOR THE THERAPEUTIC TREATMENT OF MYOPIA”. The entire contents of the above-referenced patent applications are incorporated by reference herein.

### FIELD OF THE INVENTION

The present invention relates to methods, systems, and devices for the therapeutic treatment of myopia. More specifically, the invention relates to devices and methods of treating myopia by applying a desired amount of pressure onto one or both eyeballs of a patient over a period of time.

### BACKGROUND

In a healthy human eye, light enters through the cornea and is refracted to pass through the pupil. After light enters through the pupil, it is passed through a lens which focuses the light towards the retina located at the back of the eye. The retina contains millions of nerve cells capable of converting the light to electrical signals which can be processed by the brain.

Refractive vision errors are the leading cause of vision related problems for people around the world. Besides the curvature problem of the cornea, refractive errors frequently occur when changes in the eye's length or shape affects the cornea's ability to properly focus light onto the retina. Patients suffering from refractive errors have a difficult time viewing fine details and their vision often appears blurry.

A common type of refractive error is myopia which is often called nearsightedness and affects roughly 30% of the US population. A patient suffering from myopia can usually see objects up close but has difficulty focusing on objects at a distance. Myopia occurs when the physical shape of the eye is longer in length than the normal, healthy eye. This condition causes light to be focused at a point in front of or otherwise away from the retina resulting in a blurred image.

Numerous methods have been proposed for the treatment of myopia. Patients suffering from myopia will typically wear eye glasses or contact lenses to properly shift the focus of light onto the retina. Alternative methods to treat myopia include laser surgery (e.g. LASIK) wherein a laser is used to flatten the cornea to facilitate focusing light properly onto the retina. Another option for treating nearsightedness is orthokeratology (ortho-k), also known as corneal refractive therapy. It is a non-surgical procedure that involves wearing a series of specially designed rigid contact lenses to gradually reshape the curvature of your cornea. The lenses place pressure on the cornea to flatten it. Thus changing how light entering the eye is focused.

It has been reported that varying types of pressure on the eyeball (i.e. by the eyelid) affects the shape of the cornea and impacts vision leading to refractive errors. It is further reported that reshaping the cornea by applying pressure to the eyeball can be used to treat myopia. However, currently available solutions for the corrective treatment of myopia require the use of complicated and expensive laser surgery or uncomfortable and expensive contact lenses which act as a temporary treatment of the symptoms and not the underlying condition causing this disorder. There are currently no cost effective devices or treatment methods available to treat the underlying cause of myopia and improve the vision of patients.

There is therefore a great need in the field for a method, system, and device to treat myopia from outside the eye by applying mild but sustained pressure to the eyeball and slowly restoring both the cornea and the eyeball shape overtime thereby improving the vision of patients.

### BRIEF SUMMARY OF THE INVENTION

The present invention comprises non-surgical therapeutic methods, systems, and devices for the treatment of myopia. The eyeball of a patient suffering from myopia is elongated causing the refract light to fall onto a spot in-front of the retina. Studies have shown that the reshaping of the cornea (i.e. through contact lenses or LASIK) improves the vision of patients. Methods proposed herein mainly target restoring the proper eyeball shape in order to allow the refracted light to fall on the retina, and provide a treatment regime for a patient suffering from myopia to apply a mild but sustained pressure to one or two eyeballs for a period of time. The patient may repeat this process multiple times as needed until their vision improves. The patient may reduce the frequency of this procedure to maintain the shape the eyeball to achieve long term benefit.

Also disclosed herein is a myopia correction device configured to place a desired amount of pressure against one or more eyeballs for a desired amount of time. In some embodiments, the device is in the form of goggles with an elastic strap to be worn around the head of a user thereby holding padding material against each eyeball. In some further embodiments, the device includes multiple layers of padding material which can be added or removed to adjust the level of pressure to be applied to each eyeball. By wearing the myopia correction device over a period of time, a patient may slowly reshape each eyeball and correct their vision.

### BRIEF DESCRIPTION OF THE DRAWINGS

The present invention is illustrated by way of example and not limited by the figures of the accompanying drawings, in which like references may indicate similar elements and in which:

FIG. 1A shows an example of the normal human eyeball.

FIG. 1B shows an an example of an elongated (myopic) eyeball.

FIG. 2 illustrates a front view of one example of a myopia correction device as worn by a user in accordance with embodiments presented herein.

FIG. 3 shows a side view of one example of a myopia correction device as worn by a user in accordance with embodiments presented herein.

FIG. 4 illustrates a side view of one example of a myopia correction device showing an example of an interior padding member in accordance with embodiments presented herein.

FIG. 5 shows a cross sectional view of an example of a myopia correction device in direct contact with the eye of a user in accordance with embodiments presented herein.

FIG. 6 shows a cross sectional view of an example of a myopia correction device spaced apart from the eye of a user in accordance with embodiments presented herein.

FIG. 7 shows the human eye (left side) and an example of multiple padding members of a myopia correction device in accordance with embodiments presented herein.

FIG. 8 illustrates a side perspective view of an example of a myopia correction device in accordance with embodiments presented herein.

FIG. 9 illustrates a side perspective view of an example of a myopia correction device in accordance with embodiments presented herein.

FIG. 10 shows an example of a method to correct refractive vision errors using a system and myopia correction device in accordance with embodiments presented herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New methods, systems, and devices for the treatment of myopia are described herein. In the following description, for purposes of explanation, numerous specific details are set forth in order to provide a thorough understanding of the present invention. It will be evident, however, to one skilled in the art that the present invention may be practiced without these specific details.

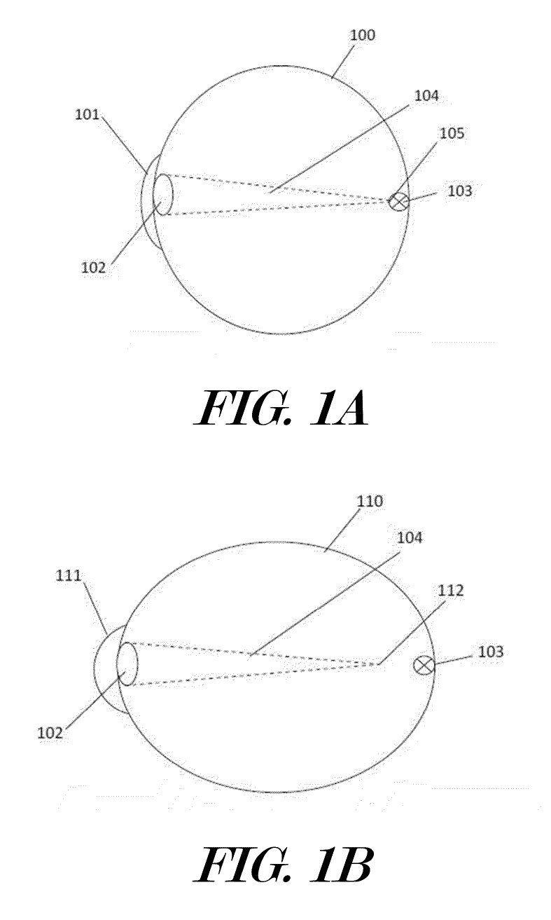

As perhaps best shown by FIG. 1A, the normal human eyeball 100 is generally more spherical in shape compared to the myopic eye (FIG. 1B) in configuration and comprises a cornea 101 on the outside of the eyeball which refracts light 104 (broken lines) entering into the eye through the pupil 102 and focuses the light onto the retina 103. In the normal eyeball 100, light 104 is focused by the cornea 101 into a spot 105 at the retina 103. The retina 103 converts the light 104 into electrical signals and transports those signals to the brain through nerve cells (not shown).

As shown in FIG. 1B, patients suffering from myopia (also called “nearsightedness”), have an eyeball 110 which is elongated to varying degrees compared to the normal eyeball 100 (FIG. 1A). Because of this elongation, the cornea 111 of the myopic eye focuses light 104 at a spot 112 in front of the retina 103 instead of onto the retina 103 causing blurred vision and other symptoms.

In some embodiments, the present invention comprises novel methods, systems, and devices of treating myopia by slowly and non-surgically reshaping the eyeballs of a user and in some embodiments the methods, systems, and devices of the invention reshape the cornea of a user. In some specific embodiments, the present invention comprises methods of applying a desired degree of pressure onto the eyeballs of a user over a period of time (also known as a “treatment schedule”). In one example, a treatment schedule may include applying a first level or degree of pressure to the right eyeball and a second level of pressure (or the same level of pressure) to the left eyeball for a period of time (i.e. overnight while the user is sleeping) (also called a “first period of time”). The user will typically wait for a second period of time (i.e. one or more days) (also called a “second period of time”) before re-applying pressure to the right and left eyeball for a period of time (i.e. overnight while the user is sleeping). In some additional embodiments, the user may increase the level of pressure applied to each eyeball at the same time or at different times. This general treatment schedule may be repeated as often as necessary to reshape the eyeballs into the proper spherical like configuration to allow the cornea to refract light directly onto the retina and thereby improving vision. Table 1 provides one non-limiting example of a possible treatment schedule according to the present disclosure:

TABLE 1

| week | Pressure Right Eye | Pressure Left Eye | Treatment Time |
|---|---|---|---|
| Week 1 | Level 1 | Level 3 | 6-9 hours nightly |
| Week 2 | Level 1 | Level 3 | 6-9 hours nightly |
| Week 3 | Level 1 | Level 3 | 6-9 hours nightly |
| Week 4 | Level 2 | Level 4 | 6-9 hours nightly |
| Week 5 | Level 2 | Level 4 | 6-9 hours nightly |
| Week 6 | Level 2 | Level 4 | 6-9 hours nightly |
| Week 7 | Level 3 | Level 5 | 6-9 hours nightly |
| Week 8 | Level 3 | Level 5 | 6-9 hours nightly |
| Week 9 | Level 3 | Level 5 | 6-9 hours nightly |
| Week 10 | Level 3 | Level 5 | 6-9 hours nightly |
| Week 11 | Level 3 | Level 5 | 6-9 hours nightly |
| Etc. | Etc. | Etc. | Etc. |

Table 1 above shows one possible example of a treatment schedule for myopia as contemplated herein. According to this example, a patient applies a first level of pressure (i.e. Level 1) to their right eyeball and a second level of pressure (i.e. Level 3) to their left eyeball for 6-9 hours each night while they are sleeping on Week 1 and increases the level of pressure on Week 4 and again on Week 7. It should be noted that the treatment schedule provided above is meant to illustrate only one example of the methods of the present invention.

In some alternative embodiments, methods of the present invention comprise applying only one fixed level of sustained pressure to each eyeball for a period of time or multiple periods of time (e.g. the levels of pressure do not change with each application).

In some embodiments, the present invention also comprises a novel apparatus to apply a desired amount of pressure onto the eyeballs of a patient suffering from myopia. In some embodiments this apparatus shall be referred to as a “myopia correction device” or simply the “device”.

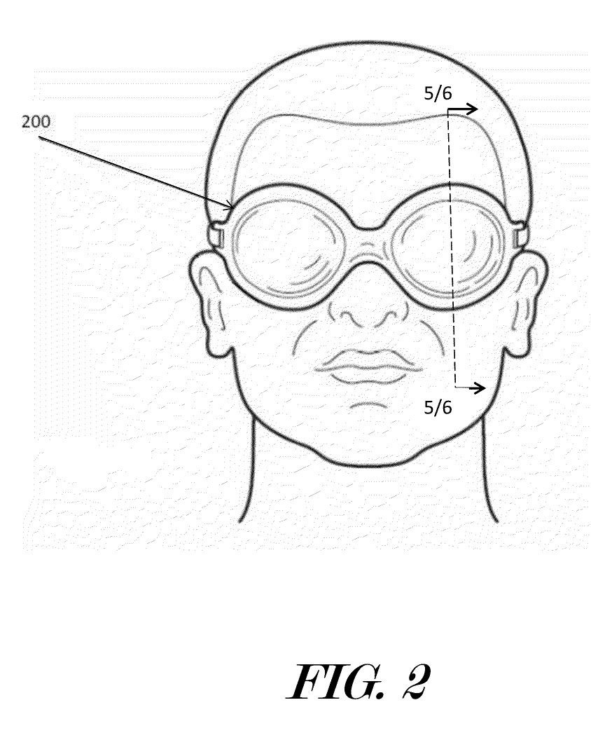

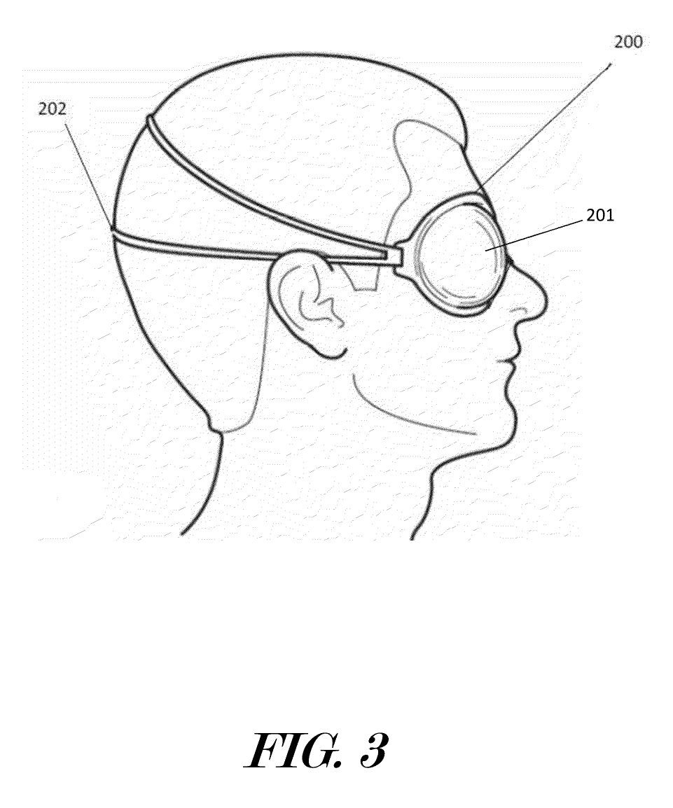

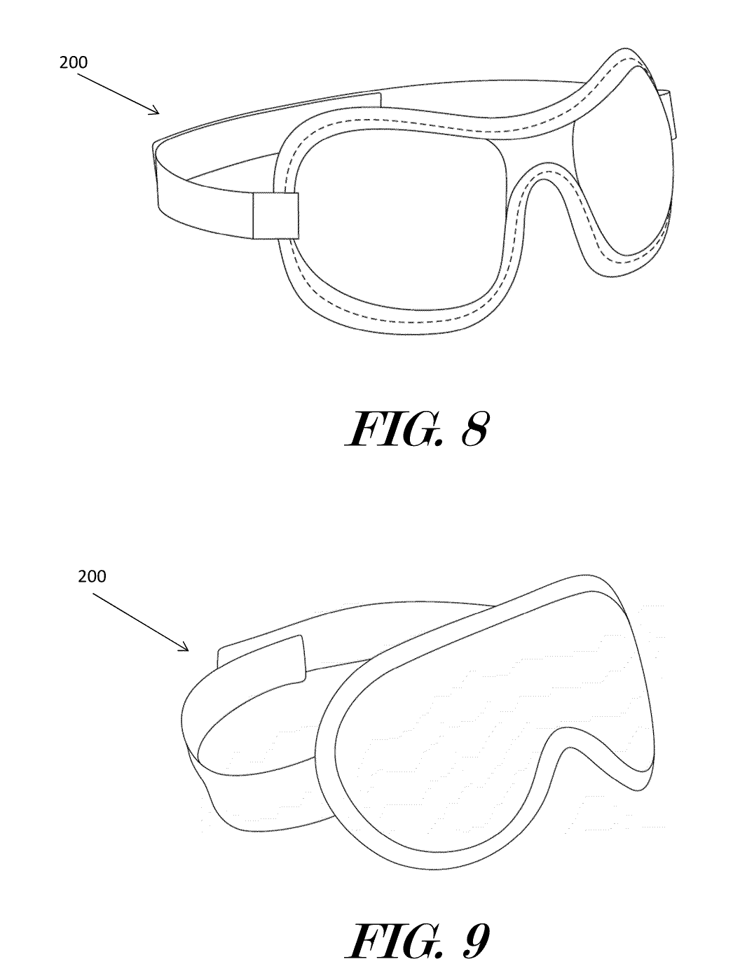

As used herein the term “myopia correction device” 200 or “device” shall preferably mean a non-invasive, non-surgical apparatus worn over the eyes of a patient suffering from myopia. The device 200 is generally configured to assert a customized level of mild pressure onto each eyeball of a person and thereby slowly changing the shape of each eyeball and in some examples each cornea as needed to correct vision. In some embodiments perhaps best shown by FIGS. 2 and 3, the myopia correction device 200 is in the form of goggles or similar design but configured to apply direct but mild pressure to one or both eyeballs. In other embodiments as perhaps best shown by FIGS. 8 and 9, the myopia correction device 200 is in the form of an eye mask, or headband, or similar design but configured to apply direct but mild pressure to one or both eyeballs. It should be noted that various other forms of myopia correction devices 200 are contemplated herein and the examples described below are meant for illustrative purposes only and should not be used to limit the scope of the invention.

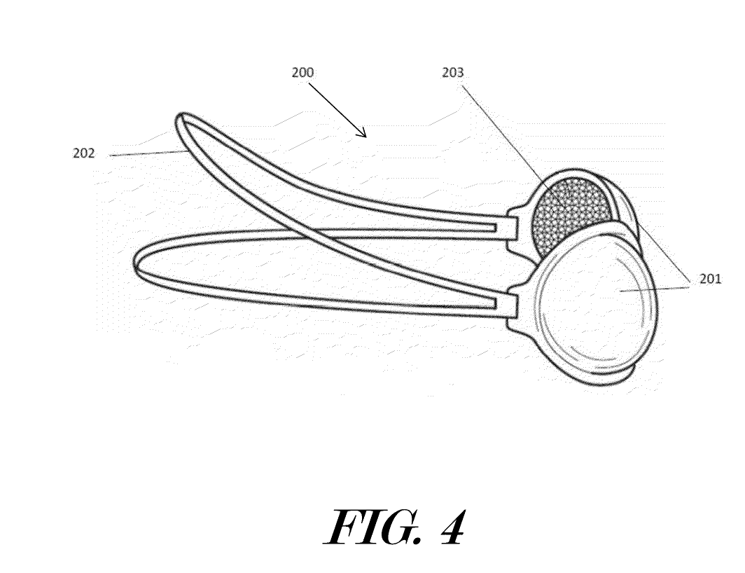

As perhaps best illustrated by FIG. 2, FIG. 3, and FIG. 4, one example of a myopia correction device 200 is shown. In this example a myopia correction device 200 is configured as a pair of eye goggles or eye mask with two opaque lens like units known as eye covers (also referred to as “eye covering members”) 201 configured to cover the eyes of a user. The eye covers 201 have an outer portion and an inner portion with said inner portion preferably configured to contain a soft or padding material such as inner padding members 203 to be placed against the eyes of a user and to assert a mild degree of pressure against each myopic eyeball through the eyelid 120.

A securing mechanism such as a strap 202 may be attached to each side of the device 200 at or near the eye covers 201 (i.e. proximate to the eye covers 201) and is configured to secure the device 200 around the head of a user. The strap 202 may be a single band or two or more bands as shown by the figures and may include adjustment and fastening mechanisms such as buckles, Velcro, snaps, and the like used to secure the strap in place and adjust it to a comfortable fit. The strap 202 may be made from an elastic material offering a secure fit around the head of a user, but not obstructive when the person is lying on a pillow.

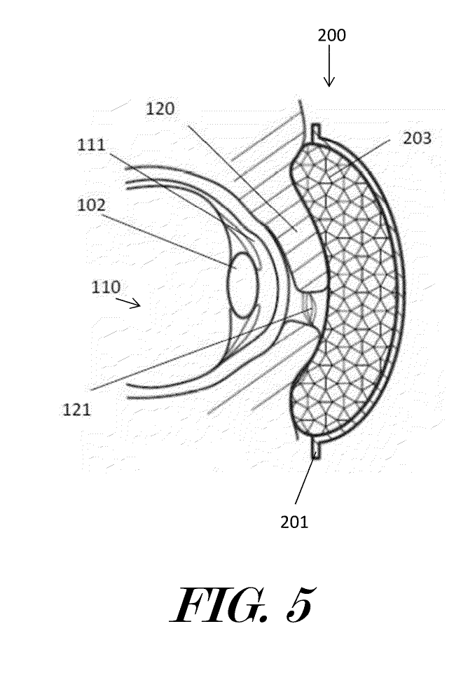

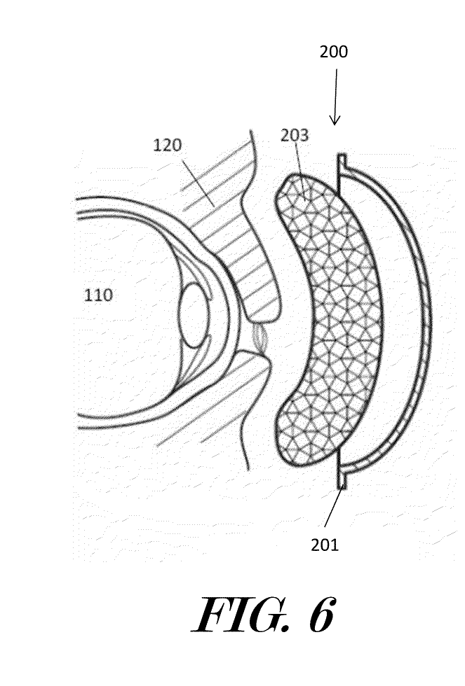

As shown by FIGS. 5 and 6, a cross sectional view (from the direction shown in FIG. 2) of an exemplary myopia correction device 200 is placed against the myopic eye 110 to apply pressure to the outside of the eyeball through the eyelid 120 and force its shape from an elongated configuration to a more spherical configuration. By changing the configuration of the eyeball, the cornea 111 may more properly focus light through the pupil 102 onto the retina (not shown). In this example, a single padding member 203 makes direct contact with the eyelid 120 and the eyelash 121 and applies pressure from outside of the eye against the exterior surface of the eyelid 120.

In some embodiments, the myopia correction device 200 comprises an eyeball pressure adjustment mechanism or eyeball pressure adjustment means to adjust the level of pressure applied to each eyeball of a person wearing the device by adjusting the tension of the strap(s) (i.e. by tightening the strap through buckles) or by adding or removing layers of padding members 203.

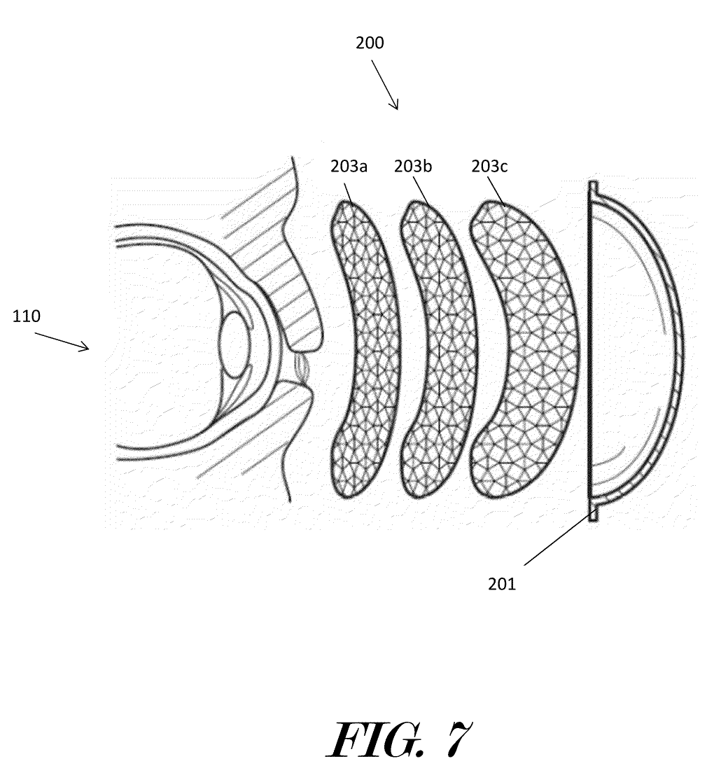

In some embodiments perhaps best shown by FIG. 7, the myopia correction device 200 and in particular the eyeball pressure adjustment mechanism comprise one, two, three, four, or even five separate pieces of padding members (known collectively as “padding members” or individually as a “padding member”) 203. The example shown in FIG. 7 provides three padding members 203 labeled as 203a, 203b, and 203c. In some embodiments, the invention comprises one or more padding members of equal size. In other embodiments, the present invention comprises one or more padding members of different sizes an in particular the thickness of each padding member 203 may be different allowing more pressure to be applied by using a padding member 203 of greater thickness or width. The padding member(s) 203 may be constructed from fabric, foam, plastic, or similar material and are configured to be rigid enough to apply pressure to the eyeball yet soft on their exterior to reduce pain or irritation when being worn by a user. The padding members 203 are further configured to be attached to the inside (facing the eyeball) of the eye covers 201 and may also be attached or connected to each other. In this embodiment, a user may adjust the degree or level of pressure asserted onto each eyeball as needed by adding or removing padding members 203. Padding members 203 may comprise a temporary attachment mechanism such as a hook and loop style fastener (i.e. Velcro) to removably connect a first padding member 203 to a second padding member 203 or a padding member 203 to the device 200.

In some cases, an eyeball with worse vision (more myopic) will require a longer treatment time and/or higher level of pressure to correct the myopic shape of that eye and thus may require more padding members 203 attached to the eye cover 201 which will be placed against that specific myopic eyeball 110.

In some further embodiments shown by FIGS. 8 and 9, the myopia correction device 200 is in the configuration of a mask commonly known as a sleep mask as known in the field. In this embodiment, the device 200 has two eye covering pieces of fabric or similar material configured to cover the eyes of a user and connected by an elastic strap to be worn around the head. The device 200 in this embodiment may include one or more padding members 203 as described above attached to the inside of the eye covering pieces and configured to be held in place against the eye of a user.

In some embodiments, the myopia correction device 200 may include a pressure applying assembly (not shown in figures) configured apply pressure onto the eyeball of a user. In this embodiment, the pressure applying assembly may take the form of a sealed elastic pocket or pouch placed on the inside (facing the eyeball) of the device 200. The sealed elastic pocked may be filled with air, liquid, or other padding materials to expand the pocket thereby applying more pressure onto the eyeball when worn by a user. In a further embodiment, the pressure applying assembly may include a gauge (not shown) to measure the level of pressure inside of the assembly to provide the user with a means to measure a desired amount of pressure to be placed against their eye when in use.

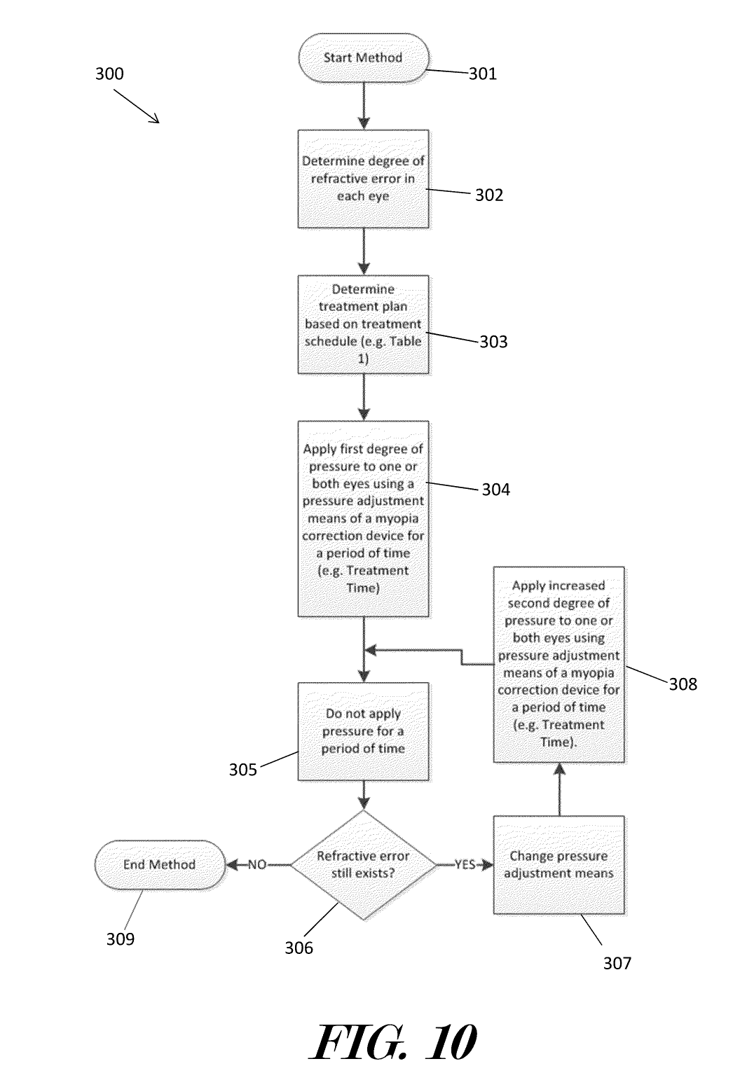

Referring now to FIG. 10, and exemplary system 300 is shown to treat refractive errors such as myopia. In this embodiment, the system 300 comprises a treatment method (FIG. 10) as well as a myopia correction device 200 (FIGS. 2-9). A user such as a patient suffering from myopia may first start the method 301 and determine the degree of refractive error in each eye 302 (i.e. by conducting a vision test either at home or at a doctor's office). The user may next determine an appropriate treatment plan based on proposed treatments schedules 303. Said treatment schedules may be updated from time to time and a sample schedule is provided herein in Table 1. To begin the treatment process, the user may select and apply a first degree of pressure to one or both eyes for a period of time (e.g. minutes or hours) 303 said degree of pressure customizable through a pressure adjustment means of a myopia correction device 200. The user may then wait without applying pressure for a period of time 305. A vision test may be conducted to determine if refractive errors still exist 306, if the errors have been corrected, the user may end the method 309. If the errors are still present, the user may change the pressure adjustment means 307 for example by adding additional padding members 203 to the myopia correction device 200. The user may then apply the increased second degree of pressure to one or both eyes for a period of time 308 using the myopia correction device 200. The user may then wait without applying pressure for a period of time 305. A vision test may be conducted to determine if refractive errors still exist 306, if the errors have been corrected, the user may end the method 309.

### NON LIMITING EXAMPLE

The following example is meant to illustrate one method and one device 200 as described herein.

According to some embodiments of the present disclosure, a user suffering from myopia may refer to a customized treatment schedule (see table 1 above for example) to select a desired amount of pressure to be placed onto each eyeball through a myopia correction device as shown by FIG. 4. The user may customize their myopia correction device 200 to assert a chosen degree of pressure to be applied to each eyeball by either adding or removing one or more padding members 203 as shown by example in FIG. 7. The user will then wear the device 200 for a first period of time (for example overnight while they are sleeping). The user may next wait for a second period of time (for example 1 day) without wearing the device 200. Next, a user may then add one or more padding members 203 to the device 200 to add more pressure to each eyeball and wear the device for a third period of time (for example overnight). The user may repeat this process and change the amount of pressure applied to each eyeball as needed to correct their vision.

### REFERENCES (INCORPORATED HEREIN BY REFERENCE)

1. Scott A. Reed et al., The Morphology of the Palpebral Fissure in Different Directions of Vertical Gaze, Optometry & Vision Science, Volume 83, Issue 10, October 2006, Pages 715-722

2. John Yee, Correcting mild myopia by means of orthoculogy, Medical Hypotheses, Volume 76, Issue 3, March 2011, Pages 332-335

I claim:

1. A myopia correction device, the device comprising:
an eye cover;
a securing mechanism configured to secure the eye cover to the head of a user; and
an eyeball pressure adjustment mechanism configured to apply varying degrees of pressure to the exterior surface of an eyeball through and eyelid.

2. The myopia correction device of claim 1 wherein the eyeball pressure adjustment mechanism contains a first padding member configured to make contact with the exterior surface of an eyelid.

3. The myopia correction device of claim 2 wherein the eyeball pressure adjustment mechanism contains a second padding member.

4. The myopia correction device of claim 3 wherein said first padding member and said second padding member are secured to each other through a temporary attachment mechanism.

5. The myopia correction device of claim 3 wherein said first padding member is of a first thickness and said second padding member is of a different second thickness.

6. The myopia correction device of claim 5 wherein the temporary attachment mechanism is a hook and loop style fastener.

7. The myopia correction device of claim 3 wherein said first and second padding members contain foam.

8. The myopia correction device of claim 1 wherein the securing mechanism is a strap.

9. The myopia correction device of claim 8 wherein the strap is made from an elastic material.

10. The myopia correction device of claim 8 wherein the strap contains an adjustment and fastening mechanisms.

11. A method to correct refractive eye errors such as myopia in a patient, the method comprising:
a) determining the level of refractive error in each eye;
b) applying a degree of pressure the exterior of the eyeball using a myopia correction device worn around the head of a patient for a first period of time;
c) waiting for a second period of time without applying pressure to the eyeball;
d) repeating steps b and c as necessary until the refractive error is corrected.

12. The method of claim 11 wherein the degree of pressure may be adjusted using a pressure adjustment mechanism of a myopia correction device.

13. The method of 12 wherein the pressure adjustment mechanism contains a first padding member configured to make contact with the exterior surface of an eyelid.

14. The method of claim 13 wherein the pressure adjustment mechanism contains a second padding member.

15. The method of claim 14 wherein said first padding member is of a first thickness and said said second padding member is of a different second thickness.

16. The method of claim 11 further comprising the step of increasing the degree of pressure using an pressure adjustment mechanism of a myopia correction device.

17. The method of 16 wherein the eyeball pressure adjustment mechanism contains a first padding member.

18. The method of 17 wherein the pressure is increased by adding a second padding member to the pressure adjustment mechanism.

19. The method of 18 wherein said first padding member and said second padding member are secured to each other through a temporary attachment mechanism.

20. A system to correct refractive eye errors such as myopia in a patient, the system comprising;
a) a myopia correction device containing a pressure adjustment mechanism;
b) a method of systemically applying pressure to the exterior of a myopic eyeball to gradually reshape the myopic eyeball; and
wherein inward pressure is applied to the exterior surface of the eyelid by the pressure adjustment mechanism of the myopia correction device worn around the head of the patient.
